(19) United States
(12) Patent Application Publication
(10) Pub. No.: US 2013/0323253 A1
(43) Pub. Date: Dec. 5, 2013

(54) XYXIN FRAGMENT BIOMARKER FOR DIAGNOSIS OF COLON CANCER

(71) Applicants: Douglas Barker, Winnipeg (CA); Katrin Atnikov, Winnipeg (CA); Yilan Zhang, Winnipeg (CA); Stephen Frost, Gurnee, IL (US)

(72) Inventors: Douglas Barker, Winnipeg (CA); Katrin Atnikov, Winnipeg (CA); Yilan Zhang, Winnipeg (CA); Stephen Frost, Gurnee, IL (US)

(73) Assignee: MIRACULINS, INC., Winnipeg, MB (CA)

(21) Appl. No.: 13/701,299

(22) PCT Filed: Jun. 1, 2011
(86) PCT No.: PCT/CA2011/050330

§ 371 Date: Jul. 12, 2013

Related U.S. Application Data
(60) Provisional application No. 61/350,195, filed on Jun. 1, 2010.

(51) Int. Cl. G01N 33/574; A61K 39/395; A61K 39/00

(52) U.S. Cl. CPC G01N 33/57419; A61K 39/0011; A61K 39/3955
USPC 424/139.1; 435/7.92; 436/501; 530/324; 530/387.9; 424/185.1

(57) ABSTRACT

The present invention relates to the field of the diagnosis of large intestine/colon diseases. More particularly, the present invention provides a method for differential diagnosis of colorectal cancer from a non-malignant disease of the large intestine/colon, and from a healthy large intestine/colon. The invention also relates to treatment of colorectal cancer.

### CROSS-REFERENCE TO RELATED APPLICATION

This application claims the benefit of, and priority, to U.S. Provisional App No. 61/350,195 filed Jun. 1, 2010 under the title “BIOMARKER FOR DIAGNOSIS AND TREATMENT OF COLORECTAL CANCER”. The content of the above-noted patent application is hereby expressly incorporated by reference into the detailed description hereof.

### FIELD OF THE INVENTION

The present invention relates to the field of the diagnosis of large intestine/colon diseases. More particularly, the present invention includes a method for differential diagnosis of colorectal cancer from a non-malignant disease of the large intestine/colon, and from a healthy large intestine/colon.

### BACKGROUND

Colorectal cancer (CRC) is the number three leading type of cancer, and the second leading cancer for estimated cancer deaths in the United States (Huang et al., 2005). In 2005, it was estimated that 149,250 new cases of CRC would be diagnosed in United States, and the estimated number of deaths as a result of CRC cancer would reach 56,290; more or less equally distributed among the genders (27,750 in women and 28,540 in men) (Cancer Facts and Figures, 2005). Overall, the incidence and mortality rates for this particular cancer are highest among individuals over the age of 50; 91% and 94% respectively (Cancer Facts and Figures, 2005). Studies have shown that the incidence of CRC is determined largely by environmental exposure. Urbanization and socio-economic status such as income level, education and access to and the quality of medical care appear to have an impact CRC incidence. North America, Europe and Australia are considered to be high-risk areas of CRC, with prevalence in countries exhibiting a Westernised lifestyle (Janout & Kollarova, 2001). Familial and hereditary factors have been observed to play primary roles in the cause of CRC. In addition, a number of other factors have been shown to be associated with an increased risk of developing CRC, such as the presence of adenomatous polyps, history/presence of inflammatory bowel disease, diet low in fibre, fruits and vegetables and high in fat and red meat, alcohol, tobacco, cholecystectomy and irradiation; while other factors such as Aspirin, NSAIDs and calcium can play a protective role (Janout & Kollarova, 2001) (Sandler, 1999).

Despite the varying hereditary or non-hereditary genetic effects linked to the development of CRC, the course of the morphological development of this cancer appears to be associated with a specific sequence of events (Wong, 2006). Typically, normal mucosa develops into an adenomatous polyp, which in some cases can progress to an adenoma with low-grade dysplasia. This type of adenoma can then, in turn, progress to a high-grade dysplasia and eventually become an invasive adenocarcinoma. Based on decades of research, the molecular mechanisms underlying these changes have been elucidated. A mutation disrupting the beta-catenin-binding region of APC gene has been shown to result in the development of early adenomas with low-grade dysplasia from the normal mucosa of the colon. Subsequently, a mutation in K-ras correlates with the progression of the early adenoma to the intermediate stage characterised by a low-grade dysplasia. This sequence of events is followed by a subsequent deleted colorectal cancer gene (DCC) mutation which promotes progression to a late adenoma with high-grade dysplasia, and then finally a p53 mutation that results in an invasive adenocarcinoma (Wong, 2006).

Despite the present knowledge of the molecular mechanisms governing the development of CRC, reliable detection methods, particularly for the early detection of the disease, are somewhat limited. Currently, the screening methods utilised by physicians include the faecal occult blood tests (FOBT), flexible sigmoidoscopy (FS), barium enema X-ray (BE), double-contrast barium enema (DCBE), colonoscopy, virtual colonoscopy (VC) and faecal DNA testing (Hendon & DiPalma, 2005) (Huang et al., 2005). Due to its relative ease, safety and cost effectiveness, the FBOT is an effective method for CRC screening (Hendon & DiPalma, 2005). Despite its effectiveness as a screening method, a major disadvantage to this test is its low diagnostic yield compared to other methods, as well as its high false-positive rate (Galiatsatos & Foulkes, 2006). Moreover, studies have brought into question whether the utilization of FOBT test can actually reduce the CRC related mortality (Hendon & DiPalma, 2005) (Moayyedi & Achkar, 2006) (Mandel et al., 1993).

In contrast, FS is a screening method that has not only been shown to reduce the mortality (Galiastsatos & Foulkes, 2006) rate related to CRC, but also to detect small polyps that are occult blood negative (Atkin et al., 1993). Like the FOBT, FS is also safe, inexpensive and cost-effective. What is more, this test can be performed without sedation (Huang et al., 2005). Unfortunately, it is able to only detect 50% of adenomas and the level of patient discomfort is compromised (Hendon & DiPalma, 2005). FS screening followed by full colonoscopy improves the detection of adenomas significantly, such that 70-80% of all advanced neoplasias can be identified (Lieberman et al., 2000). Both the BE and DCBE are also cost effective and safe, but their sensitivity is low and lack therapeutic capability (Hendon & DiPalma, 2005) (Huang et al., 2005).

In conjunction with the number of available screening methods, colonoscopy is the recommended confirmatory method for any positive findings (Huang et al., 2005) previously detected. It allows for the visualization of the entire colon and the simultaneous performance of a biopsy and a polypectomy. The disadvantages to this technique are multiple and include high costs, the use of conscious sedation thereby increasing patient recovery time following the procedure, the need for highly trained personnel, and higher complication rates as compared to other screening methods (Huang et al., 2005).

In addition, imaging technologies such as VC, derived from computed tomography (CT) has become received broader acceptance as a CRC screening tool. It requires no sedation and it is an easy, less labour-intensive screening method as compared to the barium enema and conventional colonoscopy (Huang et al. 2005) (Laghi, 2005) (Bogoni et al., 2005). Currently, the disadvantages of this screening tool involves poor sensitivity for polyp detection at less than 5 mm and a relatively high false-positive rate, which may result in an unnecessary follow-up colonoscopy (Huang et al., 2005). Moreover, its radiation dose may pose a long-term risk for screened individuals (Prokop, 2005).

Finally, faecal DNA testing is based on the understanding of the molecular events that occur during the transformation of adenomas to CRC. This particular genetic screen is a neoplasm-specific and non-invasive screening method, with no bowel preparation or dietary restrictions required. It also has the potential to detect neoplasia throughout the entire length of colon from a single collection. Its current limitations are lack of dada from screening populations and the need to confine and determine how many and which markers are necessary, as well as the necessary expenses to execute the test (about $500-$800 per test) (Huang et al., 2005).

Despite the availability of screening methods for the detection of CRC, no one method is able to detect CRC within its early stages. As a result, significant differences exist regarding the survival of patients affected by CRC according to the stages at which the disease is diagnosed (Wong, 2006). Most patients exhibit symptoms such as rectal bleeding, pain, abdominal distension or weight loss only after the disease is in its advanced stages, leaving little therapeutic options available. Diagnosis at an early stage, prior to lymph-node spread, can significantly improve the rate of survival as compared to a diagnosis established at a later stage of the disease, since the therapies used to treat colorectal cancer are stage-dependent.

Based on this, physicians and patients should discuss the advantages and disadvantages of each option when deciding which of the tests to perform. In order to reduce colorectal cancer mortality, it is suggested that people age 50 or older with no other risk factor should be screened for CRC (Huang et al., 2005) (Wong, 2006). The high-risk population, including the ones that have a family or personal history of colorectal cancer, colorectal polyps, or chronic inflammatory bowel disease, should be tested prior to the age of 50 (Cancer Facts and Figures, 2005). However, the utilization of CRC screening methods remains low. Some of the major problems from the public include a fear of being hurt by the techniques used, particularly the colonoscopy, as well as an unawareness of the necessity for screening for the disease without symptoms (Hendon & DiPalma, 2005).

Zyxin is a protein component of the focal cell adhesion plaques and is postulated to play important regulatory roles in cell to cell contact through the plasma membrane. Zyxin is widely distributed in different tissues and is present as an 84 kDa protein in western blots.

Provided herein is a new biomarker for the detection of CRC in a patient. We have identified a fragment of zyxin that can be used as a biomarker for CRC. It would be advantageous to have a new diagnostic tool for the detection of CRC in a patient, that provides higher sensitivity and specificity for the detection of CRC than other methods, a lower false-positive rate of diagnosis, and/or a reduction in the number of patients requiring further screening. It would also be advantageous to have a blood test for the detection of CRC. Finally, a fragment of zyxin can be used as a therapeutic, and to screen for other therapeutics for treatment of CRC.

### SUMMARY OF THE INVENTION

The present invention relates to methods for differential diagnosis of colorectal cancer or a non-malignant disease of the large intestine/colon by detecting a fragment of zyxin expressed within a test sample of a given subject, comparing results with samples from healthy subjects, subjects having precancerous colorectal lesion, subjects with non-malignant disease of the large intestine/colon, subjects with localized cancer of the large intestine/colon, subjects with metastasised cancer of the large intestine/colon, and/or subjects with an acute or a chronic inflammation of colorectal tissue, wherein the comparison allows for the differential diagnosis of a subject as healthy, having a precancerous colorectal lesion, having non-malignant disease of the large intestine/colon, having localized colorectal cancer, having a metastasised colorectal cancer or having an acute or chronic inflammation of colorectal tissue. A further embodiment of the present invention includes amino acids 62-110 of zyxin.

Furthermore, the invention includes kits for differential diagnosis of a non-malignant disease of the large intestine/colon and/or a localized cancer of the large intestine/colon and/or a metastasised cancer of the large intestine/colon and/or an acute or a chronic inflammation of colorectal tissue. The invention can also include kits for differential diagnosis of a subject having non-malignant disease of the large intestine/colon, a subject having precancerous colorectal lesion, a subject having localized cancer of the large intestine/colon, a subject having metastasised cancer of the large intestine/colon or a subject with an acute or a chronic inflammation of colorectal tissue. Kits can provide a sample standard comprising a biomarker of the present invention in suspension, and can also comprise instructions for the use thereof.

Test or biological samples according to the invention may be of blood, serum, plasma, urine, semen, seminal fluid, seminal plasma, pre-ejaculatory fluid (Cowper's fluid), nipple aspirate, vaginal fluid, excreta, tears, saliva, sweat, bile, biopsy, ascites, cerebrospinal fluid, lymph, or tissue extract origin. Preferably, the test and/or biological samples are urine, blood, serum, plasma and excreta samples, and are isolated from subjects of mammalian origin, preferably of human origin. Preferred test and/or biological samples are serum samples.

A further embodiment of the invention is a method for differential diagnosis of colorectal cancer and non-malignant disease of the large intestine/colon in a subject comprising obtaining a biological sample from a subject, detecting a quantity, presence, or absence of amino acids 62-100 of zyxin.

A further embodiment of the invention is a method for differential diagnosis of healthy, non-malignant disease of the large intestine/colon, precancerous colorectal lesion, localized cancer of the large intestine/colon, metastasised cancer of the large intestine/colon, and acute or chronic inflammation of colorectal tissue in a subject comprising obtaining a biological sample from a subject, detecting a quantity, presence, or absence of amino acids 62-100 of zyxin in the sample, and classifying the subject as having one of these diseases. The subject may be a mammal, for example, a human, and the biological sample or reference biological sample can be blood, serum, plasma, urine, semen, seminal fluid, seminal plasma, pre-ejaculate (Cowper's fluid), nipple aspirate, vaginal fluid, excreta, tears, saliva, sweat, biopsy, ascites, cerebrospinal fluid, lymph, or tissue extract sample.

A biologically active surface may comprise an adsorbent consisting of cationic quaternary ammonium groups.

In another aspect of the present invention, detection of a quantity, presence, or absence of a biomarker can be performed by antibody immunoassay.

In a further aspect of the present invention the antibody immunoassays can be EIA (enzyme immunoassay), RIA (radioimmunoassay), immunoprecipitation, FIA (fluorescence immunoassay), FPIA (fluorescence polorization immunoassay), CIA (chemiluminescent immunoassay), electochemical detection and these assay can be performed individually or in multiplex. Where the antibody on the solid phase can be absorbed or covalently bound to but not limited to latex beads, plastic surface, nanoparticles, magnetic particles, and other adsorbent papers such as nitrocellulose.

Another aspect of the present invention is a kit for the diagnosis of colorectal cancer within a subject comprising a biologically active surface comprising an absorbent, binding solutions, and instructions to use the kit. The absorbent may consist of cationic quaternary ammonium groups.

Another aspect of the present invention is a method for the in vitro diagnosis of a colorectal cancer in a subject comprising detection of amino acids 62-100 of zyxin in a biological sample by obtaining the biological sample from the subject, detecting the quantity, presence, or absence of the biomarker in the sample, wherein the quantity, presence or absence of the biomarker allows for the diagnosis of the subject as healthy or having colorectal cancer. Another aspect of the present invention is a method for the in vitro diagnosis of colorectal cancer and non-malignant disease of the large intestine/colon in a subject comprising detection of amino acids 62-100 of zyxin in a biological sample by obtaining the biological sample from the subject and detecting the quantity, presence, or absence of amino acids 62-100 of zyxin in the sample, wherein the quantity, presence or absence of the biomarker allows for the diagnosis of the subject as healthy, as having colorectal cancer, or as having non-malignant disease of the large intestine/colon.

Another aspect of the present invention is a method for in vitro diagnosis of colorectal cancer, non-malignant disease of the large intestine/colon, precancerous colorectal lesion, localized cancer of the large intestine/colon, metastasised cancer of the large intestine/colon, and acute or chronic inflammation of colorectal tissue in a subject comprising detecting a biomarker of amino acids 62-100 of zyxin in a biological sample by obtaining the biological sample from the subject, and detecting a quantity, presence, or absence of the biomarker in the sample, wherein the quantity, presence or absence of the biomarker allows for the diagnosis of the subject as healthy, as having colorectal cancer, non-malignant disease of the large intestine/colon, precancerous colorectal lesion, localized cancer of the large intestine/colon, metastasised cancer of the large intestine/colon, or having acute or chronic inflammation of colorectal tissue.

Another aspect of the present invention is a kit for diagnosing colorectal cancer within a subject comprising a solution, one or more binding molecules, a detection substrate, and instructions, wherein the instructions outline any of the above methods.

An aspect of the present invention is an isolated zyxin fragment of amino acids 62-100.

Another aspect of the present invention is the use of amino acids 62-100 of zyxin in the diagnosis or treatment of any of the diseases or disorders mentioned above.

Another aspect of the present invention is detecting or quantifying amino acids 62-100 of zyxin in a biological sample from a subject to determine whether the subject has colorectal cancer. The detection or quantification of amino acids 62-100 of zyxin may also be used to determine whether the subject has non-malignant disease of the large intestine/colon. In addition, the detection or quantification of amino acids 62-100 of zyxin may also be used to determine whether the subject has benign large intestine/colon disease, precancerous colorectal lesions, localized cancer of the large intestine/colon, metastasised cancer of the large intestine/colon, or acute or chronic inflammation of the large intestine/colon.

A further aspect of the present invention is a composition for treating a large intestine/colon disease comprising a molecular entity which modulates amino acids 62-100 of zyxin. Large intestine/colon disease may be colorectal cancer or non-malignant disease of the large intestine/colon. Large intestine/colon disease may be non-malignant disease of the large intestine/colon, precancerous colorectal lesion, localized cancer of the large intestine/colon, metastasised cancer of the large intestine/colon, or acute or chronic inflammation of colorectal tissue. A molecular entity may be a nucleotide, an oligonucleotide, polynucleotide, amino acid, peptide, polypeptide, protein, antibody, immunoglobulin, small organic molecule, pharmaceutical agent, agonist, antagonist, derivative, or a combination thereof.

A further aspect of the invention is a composition as described above for treating a subject having a disease of the large intestine/colon. Within the context of the invention, a disease of the large intestine/colon may be colorectal cancer or a non-malignant disease of the large intestine/colon. Large intestine/colon disease may be non-malignant disease of the large intestine/colon, precancerous colorectal lesion, localized cancer of the large intestine/colon, metastasised cancer of the large intestine/colon, or acute or chronic inflammation of colorectal tissue.

A further aspect of the present invention is a composition for treating a subject having a disease of the large intestine/colon comprising any composition identified by any of the above methods, and a pharmaceutically acceptable carrier. A disease of the large intestine/colon may be colorectal cancer or a non-malignant disease of the large intestine/colon. A disease of the large intestine/colon may also be a non-malignant disease of the large intestine/colon, a precancerous colorectal lesion, a localized cancer of the large intestine/colon, a metastasised cancer of the large intestine/colon, or an acute or chronic inflammation of colorectal tissue. A molecular entity may be a nucleotide, an oligonucleotide, polynucleotide, amino acid, peptide, polypeptide, protein, antibody, immunoglobulin, small organic molecule, pharmaceutical agent, agonist, antagonist, derivative, or a combination thereof.

Another aspect of the present invention is a use of any of the compositions described above for treating a subject having a disease of the large intestine/colon. A disease of the large intestine/colon may be colorectal cancer or a non-malignant disease of the large intestine/colon. A disease of the large intestine/colon may also be a non-malignant disease of the large intestine/colon, a precancerous colorectal lesion, a localized cancer of the large intestine/colon, a metastasised cancer of the large intestine/colon, or an acute or chronic inflammation of colorectal tissue.

### DETAILED DESCRIPTION OF THE INVENTION

Amino acids 62-100 of zyxin refers to:

(SEQ ID NO: 1)
EIPPPPPEDFPLPPPPLAGDGDDAEGALGGAFPPPPPPIEESFPPAPLE.

Amino acids 62-100 refers to the residues of the full-length human zyxin protein (Accession No. Q15942 (GI:2497677)).

(SEQ ID NO: 2)
10 20 30 40 50 60
MAAPRPSPAI SVSVSAPAFY APQKKFGPVV APKPKVNPFR PGDSEPPPAP GAQRAQMGRV
70 80 90 100 110 120
GEIPPPPPED FPLPPPPLAG DGDDAEGALG GAFPPPPPPI EESFPPAPLE EEIFPSPPPP
130 140 150 160 170 180
PEEEGGPEAP IPPPPQPREK VSSIDLEIDS LSSLLDDMTK NDPFKARVSS GYVPPPVATP
190 200 210 220 230 240
FSSKSSTKPA AGGTAPLPPW KSPSSSQPLP QVPAPAQSQT QFHVQPQPQP KPQVQLHVQS
250 260 270 280 290 300
QTQPVSLANT QPRGPPASSP APAPKFSPVT PKFTPVASKF SPGAPGGSGS QPNQKLGHPE
310 320 330 340 350 360
ALSAGTGSPQ PPSFTYAQQR EKPRVQEKQH PVPPPAQNQN QVRSPGAPGP LTLKEVEELE
370 380 390 400 410 420
QLTQQLMQDM EHPQRQNVAV NELCGRCHQP LARAQPAVRA LGQLFHIACF TCHQCAQQLQ
430 440 450 460 470 480
GQQFYSLEGA PYCEGCYTDT LEKCNTCGEP ITDRMLRATG KAYHPHCFTC VVCARPLEGT
490 500 510 520 530 540
SFIVDQANRP HCVPDYHKQY APRCSVCSEP IMPEPGRDET VRVVALDKNF HMKCYKCEDC
550 560 570
GKPLSIEADD NGCFPLDGHV LCRKCHTARA QT

The term “biomolecule” refers to a molecule that is produced by a cell or tissue in an organism. Such molecules include, but are not limited to, molecules comprising nucleic acids, nucleotides, oligonucleotides, polynucleotides, amino acids, peptides, polypeptides, proteins, monoclonal and/or polyclonal antibodies, antigens, sugars, carbohydrates, fatty acids, lipids, steroids, and combinations thereof (e.g., glycoproteins, ribonucleoproteins, lipoproteins). Furthermore, the terms “nucleotide”, “oligonucleotide” or polynucleotide” refer to DNA or RNA of genomic or synthetic origin which may be single-stranded or double-stranded and may represent the sense or the antisense strand. Included as part of the definition of “oligonucleotide” or “polynucleotide” are peptide polynucleotide sequences (e.g., peptide nucleic acids; PNAs), or any DNA-like or RNA-like material (e.g., morpholinos, ribozymes).

The term “molecular entity” refers to any defined inorganic or organic molecule that is either naturally occurring or is produced synthetically. Such molecules include, but are not limited to, biomolecules as described above, simple and complex molecules, acids and alkalis, alcohols, aldehydes, arenas, amides, amines, esters, ethers, ketones, metals, salts, and derivatives of any of the aforementioned molecules.

The term “fragment” refers to a portion of a polynucleotide or polypeptide sequence that comprises at least 15 consecutive nucleotides or 5 consecutive amino acid residues, respectively.

The terms “biological sample” and “test sample” refer to all biological fluids and excretions isolated from any given subject. In the context of the invention such samples include, but are not limited to, blood, serum, plasma, urine, semen, seminal fluid, seminal plasma, pre-ejaculatory fluid (Cowper's fluid), nipple aspirate, vaginal fluid, excreta, tears, saliva, sweat, biopsy, ascites, cerebrospinal fluid, lymph, marrow, hair or tissue extract samples.

The term “specific binding” refers to the interaction between two biomolecules that occurs under specific conditions. The binding of two biomolecules is considered to be specific when the interaction between said molecules is substantial. Moreover, the phrase “specific conditions” refers to reaction conditions that permit, enable, or facilitate the binding of said molecules such as pH, salt, detergent and other conditions known to those skilled in the art.

The term “interaction” relates to the direct or indirect binding or alteration of biological activity of a biomolecule.

The term “differential diagnosis” refers to a diagnostic decision between healthy and different disease states, including various stages of a specific disease. A subject is diagnosed as healthy or to be suffering from a specific disease, or a specific stage of a disease based on a set of hypotheses that allow for the distinction between healthy and one or more stages of the disease. The choice between healthy and one or more stages of disease depends on a significant difference between each hypothesis. Under the same principle, a “differential diagnosis” may also refer to a diagnostic decision between one disease type as compared to another (e.g. colorectal cancer vs. a non-malignant disease of the large intestine/colon).

The term “colorectal cancer” refers to a malignant neoplasm of the large intestine/colon within a given subject, wherein the neoplasm is of epithelial origin and is also referred to as a carcinoma of the large intestine/colon. According to the invention, colorectal cancer is defined according to its type, stage and/or grade. Typical staging systems known to those skilled in the art such as the Gleason Score (a measure of tumour aggressiveness based on pathological examination of tissue biopsy), the Jewett-Whitmore system and the TNM system (the system adopted by the American Joint Committee on Cancer and the International Union Against Cancer). The term “colorectal cancer”, when used without qualification, includes both localized and metastasised colorectal cancer. The term “colorectal cancer” can be qualified by the terms “localized” or “metastasised” to differentiate between different types of tumour as those words are defined herein. The terms “colorectal cancer” and “malignant disease of the large intestine/colon” are used interchangeably herein.

The terms “neoplasm” or “tumour” may be used interchangeably and refer to an abnormal mass of tissue wherein growth of the mass surpasses and is not coordinated with the growth of normal tissue. A neoplasm or tumour may be defined as “benign” or “malignant” depending on the following characteristics: degree of cellular differentiation including morphology and functionality, rate of growth, local invasion and metastasis. A “benign” neoplasm is generally well differentiated, has characteristically slower growth than a malignant neoplasm and remains localised to the site of origin. In addition a benign neoplasm does not have the capacity to infiltrate, invade or metastasise to distant sites. A “malignant” neoplasm is generally poorly differentiated (anaplasia), has characteristically rapid growth accompanied by progressive infiltration, invasion and destruction of the surrounding tissue. Furthermore, a malignant neoplasm has to capacity to metastasise to distant sites.

The term “differentiation” refers to the extent to which parenchymal cells resemble comparable normal cells both morphologically and functionally.

The term “metastasis” refers to the spread or migration of cancerous cells from a primary (original) tumour to another organ or tissue, and is typically identifiable by the presence of a “secondary tumour” or “secondary cell mass” of the tissue type of the primary (original) tumour and not of that of the organ or tissue in which the secondary (metastatic) tumour is located. For example, a colorectal cancer that has migrated to bone is said to be metastasised colorectal cancer, and consists of cancerous colorectal cancer cells in the large intestine/colon as well as cancerous colorectal cancer cells growing in bone tissue.

The terms “non-malignant disease of the large intestine/colon”, “non-colorectal cancer state” and “benign colorectal disease” may be used interchangeably and refer to a disease state of the large intestine/colon that has not been classified as colorectal cancer according to specific diagnostic methods including but not limited to rectal palpitation, PSA scoring, transrectal ultrasonography and tissue biopsy. Such diseases include, but are not limited to an inflammation of colorectal tissue (e.g., Inflammatory Bowel Disease including Crohn's disease and ulcerative colitis) and benign large intestine/colon hyperplasia.

The term “healthy” refers to a subject possessing good health. Such a subject demonstrates an absence of any malignant or non-malignant disease of the large intestine/colon. In the context of this application, a “healthy individual” is only healthy in that they have an absence of any malignant or non-malignant disease of the large intestine/colon; a “healthy individual” may have other diseases or conditions that would normally not be considered “healthy”.

The phrase “pre-cancerous lesion of the large intestine/colon” or “precancerous large intestine/colon lesion” refers to a biological change within the large intestine/colon such that it becomes susceptible to the development of a malignant neoplasm. More specifically, a pre-cancerous lesion of the large intestine/colon is a preliminary stage of a colorectal cancer. Causes of a pre-cancerous lesion may include, but are not limited to, genetic predisposition and exposure to cancer-causing agents (carcinogens); such cancer causing agents include agents that cause genetic damage and induce neoplastic transformation of a cell.

The phrase “neoplastic transformation of a cell” refers an alteration in normal cell physiology and includes, but is not limited to, self-sufficiency in growth signals, insensitivity to growth-inhibitory (anti-growth) signals, evasion of programmed cell death (apoptosis), limitless replicative potential, sustained angiogenesis, and tissue invasion and metastasis.

The term “differentially present” refers to differences in the quantity of a biomolecule present in samples taken from colorectal cancer patients as compared to samples taken from subjects having a non-malignant disease of the large intestine/colon or healthy subjects. Furthermore, a biomolecule is differentially present between two samples if the quantity of said biomolecule in one sample population is significantly different (defined statistically) from the quantity of said biomolecule in another sample population. For example, a given biomolecule may be present at elevated, decreased, or absent levels in samples of taken from subjects having colorectal cancer compared to those taken from subjects who do not have a colorectal cancer.

The term ‘biological activity’ may be used interchangeably with the terms ‘biologically active’, ‘bioactivity’ or ‘activity’ and, for the purposes herein, means an effector or antigenic function that is directly or indirectly performed by a biomarker of the invention (whether in its native or denatured conformation), derivative or fragment thereof. Effector functions include phosphorylation (kinase activity) or activation of other molecules, induction of differentiation, mitogenic or growth promoting activity, signal transduction, immune modulation, DNA regulatory functions and the like, whether presently known or inherent. Antigenic functions include possession of an epitope or antigenic site that is capable of cross-reacting with antibodies raised against a naturally occurring or denatured biomarker of the invention, derivative or fragment thereof. Accordingly, a biological activity of such a protein can be that it functions as regulator of a signalling pathway of a target cell. Such a signalling pathway can, for example, modulate cell differentiation, proliferation and/or migration of such a cell, as well as tissue invasion, tumour development and/or metastasis. A target cell according to the invention can be a neoplastic or cancer cell.

The terms ‘neoplastic cell’ and ‘neoplastic tissue’ refer to a cell or tissue, respectively, that has undergone significant cellular changes (transformation). Such cellular changes are manifested by an escape from specific control mechanisms, increased growth potential, alteration in the cell surface, karyotypic abnormalities, morphological and biochemical deviations from the norm, and other attributes conferring the ability to invade, metastasize and kill.

The term “diagnostic assay” can be used interchangeably with “diagnostic method” and refers to the detection of the presence or nature of a pathologic condition.

Within the context of the invention, the term “true positives” refers to those subjects having a localized or a metastasised cancer of the large intestine/colon or a benign large intestine/colon disease, a precancerous colorectal lesion, or an acute or a chronic inflammation of colorectal tissue and who are categorized as such by the diagnostic assay. Depending on context, the term “true positives” may also refer to those subjects having either colorectal cancer or a non-malignant disease of the large intestine/colon, who are categorized as such by the diagnostic assay.

Within the context of the invention, the term “false negatives” refers to those subjects having either a localized or a metastasised cancer of the large intestine/colon, a benign large intestine/colon disease, a precancerous colorectal lesion, or an acute or a chronic inflammation of colorectal tissue and who are not categorized as such by the diagnostic assay. Depending on context, the term “false negatives” may also refer to those subjects having either colorectal cancer or a non-malignant disease of the large intestine/colon and who are not categorized as such by the diagnostic assay.

Within the context of the invention, the term “true negatives” refers to those subjects who do not have a localized or a metastasised cancer of the large intestine/colon, a benign large intestine/colon disease, a precancerous colorectal lesion, or an acute or a chronic inflammation of colorectal tissue and who are categorized as such by the diagnostic assay. Depending on context, the term “true negatives” may also refer to those subjects who do not have colorectal cancer or a non-malignant disease of the large intestine/colon and who are categorized as such by the diagnostic assay.

Within the context of the invention, the term “false positives” refers to those subjects who do not have a localized or a metastasised cancer of the large intestine/colon, a benign large intestine/colon disease, a precancerous colorectal lesion, or an acute or a chronic inflammation of colorectal tissue but are categorized by the diagnostic assay as having a localized or metastasised cancer of the large intestine/colon, a benign large intestine/colon disease, a precancerous colorectal lesion or an acute or chronic inflammation of colorectal tissue. Depending on context, the term “false positives” may also refer to those subjects who do not have colorectal cancer or a non-malignant disease of the large intestine/colon but are categorized by the diagnostic assay as having colorectal cancer or a non-malignant disease of the large intestine/colon.

The term “sensitivity”, as used herein in the context of its application to diagnostic assays, refers to the proportion of all subjects with localized or metastasised cancer of the large intestine/colon, a benign large intestine/colon disease, a precancerous colorectal lesion, or an acute or a chronic inflammation of colorectal tissue that are correctly identified as such (that is, the number of true positives divided by the sum of the number of true positives and false negatives).

The term “specificity” of a diagnostic assay, as used herein in the context of its application to diagnostic assays, refers to the proportion of all subjects with neither localized or metastasised cancer of the large intestine/colon nor a benign large intestine/colon disease, a precancerous colorectal lesion, or an acute or a chronic inflammation of colorectal tissue that are correctly identified as such (that is, the number of true negatives divided by the sum of the number of true negatives and false positives).

The term “adsorbent” refers to any material that is capable of accumulating (binding) a given biomolecule. The adsorbent typically coats a biologically active surface and is composed of a single material or a plurality of different materials that are capable of binding a biomolecule. Such materials include, but are not limited to, anion exchange materials, cation exchange materials, metal chelators, polynucleotides, oligonucleotides, peptides, antibodies, naturally occurring compounds, synthetic compounds, etc.

The phrase “biologically active surface” refers to any two- or three-dimensional extensions of a material that biomolecules can bind to, or interact with, due to the specific biochemical properties of this material and those of the biomolecules. Such biochemical properties include, but are not limited to, ionic character (charge), hydrophobicity, or hydrophilicity.

The term “binding biomolecule” refers to a molecule that displays an affinity for another biomolecule.

The term “immunogen” may be used interchangeably with the phrase “immunising agent” and refers to any substance or organism that provokes an immune response when introduced into the body of a given subject. All immunogens are considered as antigens and, in the context of the invention, can be defined on the basis of their immunogenicity, wherein “immunogenicity” refers to the ability of the immunogen to induce either a humoral or a cell-mediated immune response. In the context of the invention an immunogen that induces a “humoral immune response” activates antibody production and secretion by cells of the B-lymphocyte lineage (B-cells) and thus can be used to for antibody production as described herein. Such immunogens may be polysaccharides, proteins, lipids or nucleic acids, or they may be lipids or nucleic acids that are complexed to either a polysaccharide or a protein. The term “solution” refers to a homogeneous mixture of two or more substances. Solutions may include, but are not limited to buffers, substrate solutions, elution solutions, wash solutions, detection solutions, standardisation solutions, chemical solutions, solvents, etc.

The phrase “coupling buffer” refers to a solution that is used to promote covalent binding of biomolecules to a biological surface.

The phrase “blocking buffer” refers to a solution that is used to (prevent) block unbound binding sites of a given biological surface from interacting with biomolecules in an unspecific manner.

The term “chromatography” refers to any method of separating biomolecules within a given sample such that the original native state of a given biomolecule is retained. Separation of a biomolecule from other biomolecules within a given sample for the purpose of enrichment, purification and/or analysis, may be achieved by methods including, but not limited to, size exclusion chromatography, ion exchange chromatography, hydrophobic and hydrophilic interaction chromatography, metal affinity chromatography, wherein “metal” refers to metal ions (e.g. nickel, copper, gallium, zinc, iron or cobalt) of all chemically possible valences, or ligand affinity chromatography wherein “ligand” refers to binding molecules, preferably proteins, antibodies, or DNA. Generally, chromatography uses biologically active surfaces as adsorbents to selectively accumulate certain biomolecules.

The phrase “mass spectrometry” refers to a method comprising employing an ionization source to generate gas phase ions from a biological entity of a sample presented on a biologically active surface, and detecting the gas phase ions with an ion detector. Comparison of the time the gas phase ions take to reach the ion detector from the moment of ionisation with a calibration equation derived from at least one molecule of known mass allows the calculation of the estimated mass to charge ratio of the ion being detected.

The phrases “mass to charge ratio”, “m/z ratio” or “m/z” can be used interchangeably and refer to the ratio of the molecular weight (grams per mole) of an ion detected by mass spectrometry to the number of charges the ion carries. Thus a single biomolecule can be assigned more than one mass to charge ratio by a mass spectrometer if that biomolecule can be ionised into more than one species each of which carries a different number of charges.

The acronym “TOF” refers to the time-of-flight of a biomolecule or other molecular entity, and particularly that of an ion in a time-of-flight type mass spectrometer. TOF values are derived by measuring the duration of flight of an ion, typically between its entry into and exit from a time-of-flight analyser tube. Alternatively, the accuracy of TOF values can be improved by methods known to those skilled in the art, for example through the use of reflectrons and/or pulsed-laser ionization. TOF values for a given ion can be applied to previously established calibration equations derived from the TOF values for ions of known mass in order to calculate the mass to charge ratio of these ions.

The phrase “laser desorption mass spectrometry” refers to a method comprising the use of a laser as an ionization source to generate gas phase ions from a biomolecule presented on a biologically active surface, and detecting the gas phase ions with a mass spectrometer.

The term “mass spectrometer” refers to a gas phase ion spectrometer that includes an inlet system, an ionisation source, an ion optic assembly, a mass analyser, and a detector.

Within the context of the invention, the terms “detect”, “detection” or “detecting” refer to the identification of the presence, absence, or quantity of a given biomolecule.

The phrase “Mann-Whitney Rank Sum Test” refers to a non-parametric statistical method used to test the null hypothesis that two sets of values that do not have normal distributions are derived from the same population.

The phrase “energy absorbing molecule” and its acronym “EAM” refers to a molecule that absorbs energy from an energy source in a mass spectrometer thereby enabling desorption of a biomolecule from a biologically active surface. Cinnamic acid derivatives, sinapinic acid and dihydroxybenzoic acid, ferulic acid and caffeic acid are frequently used as energy-absorbing molecules in laser desorption of biomolecules. See U.S. Pat. No. 5,719,060 (Hutchens & Yip) for a further description of energy absorbing molecules.

The terms “peak” and “signal” may be used interchangeably, and refer to a defined, non-background value which is generated by a population of a given biomolecule of a certain molecular mass that has been ionised contacting the detector of a mass spectrometer, wherein the size of the population can be roughly related to the degree of the intensity of the signal Typically, this “signal” can be defined by two values: an apparent mass-over-charge ratio (m/z) and an intensity value generated as described.

The phrases “peak intensity”, “intensity of a peak” and “intensity” may be used interchangeably, and refer to the relative amount of a biomolecule contacting the detector of a mass spectrometer in relation to other peaks in the same mass profile. Typically, the intensity of a peak is expressed as the maximum observed signal within a defined mass range that adequately defines the peak.

The phrases “signal to noise ratio”, “SN ratio” and “SN” may be used interchangeably, and refer to the ratio of a peak's intensity and a dynamically calculated value representing the average background signal detected in the approximate mass range of the peak. The SN ratio of a peak is typically used as an objective criterion for (a) computer-assisted peak detection and/or (b) manual evaluation of a peak as being an artefact.

The term “cluster” refers to a peak that is present in a certain set of mass spectra or mass profiles obtained from different samples belonging to two or more different groups (e.g. subjects with colorectal cancer and healthy subjects). Within the set of spectra, the peaks or signals belonging to a given cluster can differ in their intensities, but not in the apparent molecular masses.

The term “classifier” refers to an algorithm or methodology which is using one or more defined traits or attributes to subdivide a population individual patients or samples or elements of data into a finite number of groups with as great a degree of accuracy as possible. The term “tree” refers to a type of classifier consisting of a branching series of decision points (typically referred to as “leaves” or “nodes”) that eventually lead to the classification of individual patients or samples or elements of data from a population into one of a finite number of groups.

The phrase “mass profile” refers to a series of discrete, non-background noise peaks that are defined by their mass to charge ratio and are characteristic of an individual mass spectrum.

The acronym “ROC-AUC” refers to the area under a receiver operator characteristic curve. This is a widely accepted measure of diagnostic utility of some tool, taking into account both the sensitivity and specificity of the tool. Typically, ROC-AUC ranges from 0.5 to 1.0, where a value of 0.5 indicates the tool has no diagnostic value and a value of 1.0 indicates the tool has 100% sensitivity and 100% specificity.

The term “sensitivity” refers to the proportion of patients with the outcome in whom the results of the decision rule are abnormal. Typically, the outcome is disadvantageous to the patient. The term “specificity” refers to the proportion of patients without the outcome in whom the results of the decision rule are normal.

The term “antibody immunoassay” refers to any analytical test that can generate a signal from an analyte present in a biological liquid, typically serum or urine, by using antibodies complementary to the antigens present on the analyte. Antibodies are very selective and only bind to their specific target, even in the presence of a multitude of alternative proteins or materials in a sample. Generally, an antibody immobilized onto a surface, usually a microtiter plate, captures the teat analyte from the sample and a different antibody, specifice for another part of the analyte, binds and acts as the detector molecule. The signal output by the detector antibody is proportional to the amount of analyte in the sample; the concentration of analyte can be quantified by comparing signal outputs to those of known standard concentrations.

The term “antibody” is used in the broadest sense and specifically includes monoclonal antibodies (including full length monoclonal antibodies), multispecific antibodies (e.g., bispecific antibodies), and antibody fragments that exhibit a desired biological activity or function. Antibodies can be chimeric, humanized, or mammalian, including mouse or human. Antibodies can also be an antibody fragment.

“Antibody fragments” comprise a portion of a full length antibody, generally the antigen binding or variable region thereof. Examples of antibody fragments include Fab, Fab′, F(ab)₂, and Fv fragments; diabodies; linear antibodies; single-chain antibody molecules; and multispecific antibodies formed from antibody fragments. “Functional fragments” substantially retain binding to an antigen of the full length antibody, and retain a biological activity.

It should be noted that as used herein and in the appended claims, the singular forms “a,” “an,” and “the” include plural reference unless the context clearly dictates otherwise. Thus, for example, a reference to “an antibody” is a reference to one or more antibodies and derivatives thereof known to those skilled in the art, and so forth.

It is to be understood that the present invention is not limited to the particular materials and methods described or equipment, as these may vary.

### Use as a Diagnostic Tool

The present invention relates to methods for the differential diagnosis of colorectal cancer or a non-malignant disease of the large intestine/colon by detecting amino acids 62-100 of zyxin within a biological sample of a given subject, comparing results with samples from healthy subjects, subjects having a non-malignant disease of the large intestine/colon and subjects having colorectal cancer, wherein the comparison allows for the differential diagnosis of a subject as healthy, having non-malignant disease of the large intestine/colon or having colorectal cancer. We have shown that patients having CRC display a significantly lower amount of the 62-100 aa fragment of zyxin in their plasma or urine as compared to a healthy individual or an individual having a non-malignant disease of the large intestine/colon.

In one aspect of the invention, a method for the differential diagnosis of colorectal cancer or a non-malignant disease of the large intestine/colon comprises: obtaining a biological sample from a given subject, contacting said sample with an adsorbent present on a biologically active surface under specific binding conditions, allowing the biomolecule within the biological sample to bind to said adsorbent, detecting one or more bound biomolecules using a detection method, wherein the detection method generates a mass profile of said sample, transforming the mass profile generated into a computer-readable form, and comparing the mass profile of said sample with a database containing mass profiles from comparable samples specific for healthy subjects, subjects having colorectal cancer, and/or subjects having a non-malignant disease of the large intestine/colon. The outcome of said comparison will allow for the determination of whether the subject from which the biological sample was obtained, is healthy, has a non-malignant disease of the large intestine/colon and/or colorectal cancer based on the presence, absence or comparative quantity of specific biomolecules. The level of the zyxin fragment (SEQ ID NO:1) can be determined by actual comparison to a sample from a healthy individual, or it can be determined quantitatively and compared to a known average quantitative result or range from healthy individuals. Alternatively, the level of the zyxin fragment (SEQ ID NO:1) can be compared in time in the same individual, to measure progression of disease or impact of treatment of the disease in that individual over time.

In one aspect of the invention, the biomolecule of the invention is amino acids 62-100 of zyxin and may be used individually to diagnose a subject as being healthy, or having a non-malignant disease of the large intestine/colon, or having a precancerous colorectal lesion, or having a localized cancer of the large intestine/colon, or having a metastasised cancer of the large intestine/colon, or having an acute or a chronic inflammation of colorectal tissue.

In yet another aspect of the invention, amino acids 62-100 of zyxin may be used in combination with another diagnostic tool to diagnose a subject as being healthy, or having a non-malignant disease of the large intestine/colon, or having a precancerous colorectal lesion, or having a localized cancer of the large intestine/colon, or having a metastasised cancer of the large intestine/colon, or having an acute or a chronic inflammation of colorectal tissue. For example, amino acids 62-100 of zyxin may be used in combination with other diagnostic tools specific for colorectal cancer detection such as, but not limited to, large intestine/colon specific antigen (PSA) testing, DRE, rectal palpitation, biopsy evaluation using Gleason scoring, radiography and symptomological evaluation by a qualified clinician.

The methods for detecting amino acids 62-100 of zyxin have many applications. For example, amino acids 62-100 of zyxin can be measured to differentiate between healthy subjects, subjects having a non-malignant disease of the large intestine/colon, subjects having a precancerous colorectal lesion, or subjects having a localized cancer of the large intestine/colon, or subjects having a metastasised cancer of the large intestine/colon, or subjects with an acute or a chronic inflammation of colorectal tissue, and thus are useful as an aid in the diagnosis of a non-malignant disease of the large intestine/colon, or a precancerous colorectal lesion, or a localized cancer of the large intestine/colon, or a metastasised cancer of the large intestine/colon, or an acute or a chronic inflammation of colorectal tissue. Alternatively, said biomolecule may be used to diagnose a subject as being healthy.

In another aspect of the invention, an in vitro binding assay can be used to detect amino acids 62-100 of zyxin within a biological sample of a given subject. A given biomolecule of the invention can be detected within a biological sample by contacting the biological sample from a given subject with specific binding molecule(s) under conditions conducive for an interaction between the given binding molecule(s) and amino acids 62-100 of zyxin. If a given biomolecule is present in the biological sample, it will form a complex with its binding molecule. To determine if the quantity of the detected biomolecule in a biological sample is comparable to a given quantity for healthy subjects, subjects having a non-malignant disease of the large intestine/colon, subjects having a precancerous colorectal lesion, subjects having a localized cancer of the large intestine/colon, subjects having a metastasised cancer of the large intestine/colon or subjects with an acute or a chronic inflammation of colorectal tissue, an amount of a complex formed between the binding molecule and amino acids 62-100 of zyxin can be determined by comparing to a standard. For example, if the amount of the complex falls within a quantitative value for healthy subjects, then the sample can be considered to be obtained from a healthy subject. If the amount of the complex falls within a quantitative value for subjects known to have a non-malignant disease of the large intestine/colon, then the sample can be considered to be obtained from a subject having a non-malignant disease of the large intestine/colon. If the amount of the complex falls within a quantitative range for subjects known to have colorectal cancer, then the sample can be considered to have been obtained from a subject having colorectal cancer. In vitro binding assays that are included within the scope of the invention are those known to the skilled in the art (e.g., ELISA, western blotting).

Thus the invention includes a method for differential diagnosis of a colorectal cancer or a non-malignant disease of the large intestine/colon comprising: detecting amino acids 62-100 of zyxin within a given biological sample. This method comprises obtaining a biological sample from a subject, contacting said sample with a binding molecule specific for a differentially expressed biomolecule, detecting an interaction between the binding molecule and its specific biomolecule, wherein the detection of an interaction indicates the presence or absence of said biomolecule, thereby allowing for the differential diagnosis of a subject as healthy, or having a non-malignant disease of the large intestine/colon, or having a precancerous colorectal lesion, or having a localized cancer of the large intestine/colon, or having a metastasised cancer of the large intestine/colon, or having an acute or a chronic inflammation of colorectal tissue. Binding molecules include, but are not limited to, nucleic acids, nucleotides, oligonucleotides, polynucleotides, amino acids, peptides, polypeptides, proteins, monoclonal and/or polyclonal antibodies, antigens, sugars, carbohydrates, fatty acids, lipids, steroids, or combinations thereof. (e.g. glycoproteins, ribonucleoproteins, lipoproteins), compounds or synthetic molecules. Preferably, binding molecules are antibodies specific for amino acids 62-100 of zyxin The biomolecules detected using the above-mentioned binding molecules include, but are not limited to, molecules comprising nucleic acids, nucleotides, oligonucleotides, polynucleotides, amino acids, peptides, polypeptides, proteins, monoclonal and/or polyclonal antibodies, antigens, sugars, carbohydrates, fatty acids, lipids, steroids, and combinations thereof (e.g., glycoproteins, ribonucleoproteins, lipoproteins). Preferably, biomolecules that are detected using the above-mentioned binding molecules include nucleic acids, nucleotides, oligonucleotides, polynucleotides, amino acids, peptides, polypeptides, proteins, monoclonal and/or polyclonal antibodies. Even more preferred are binding molecules that are amino acids, peptides, polypeptides, proteins, monoclonal and/or polyclonal antibodies.

For example, in vivo, antibodies or fragments thereof may be utilised for the detection of the polypeptide fragment containing amino acids 62-110 of zyxin in a biological sample, for example, by applying a labelled antibody directed against amino acids 62-110 of zyxin to said biological sample under conditions that favour an interaction between the labelled antibody and its corresponding biomolecule. Depending on the nature of the biological sample, it is possible to determine not only the presence of a biomolecule, but also its cellular distribution. For example, in a blood serum sample, only the serum levels of a given biomolecule can be detected, whereas its level of expression and cellular localisation can be detected in histological samples. It will be obvious to those skilled in the art, that a wide variety of methods can be modified in order to achieve such detection.

In another example, an antibody directed against a biomolecule of the invention that is coupled to an enzyme is detected using a chromogenic substrate that is recognised and cleaved by the enzyme to produce a chemical moiety, which is readily detected using spectrometric, fluorimetric or visual means. Enzymes used to for labelling include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Detection may also be accomplished by visual comparison of the extent of the enzymatic reaction of a substrate with that of similarly prepared standards. Alternatively, radiolabelled antibodies can be detected using a gamma or a scintillation counter, or they can be detected using autoradiography. In another example, fluorescently labelled antibodies are detected based on the level at which the attached compound fluoresces following exposure to a given wavelength. Fluorescent compounds typically used in antibody labelling include, but are not limited to, fluorescein isothiocynate, rhodamine, phycoerthyrin, phycocyanin, allophycocyani, o-phthaldehyde and fluorescamine. In yet another example, antibodies coupled to a chemi- or bioluminescent compound can be detected by determining the presence of luminescence. Such compounds include, but are not limited to, luminal, isoluminal, theromatic acridinium ester, imidazole, acridinium salt, oxalate ester, luciferin, luciferase and aequorin.

Furthermore, in vivo techniques for detecting a biomolecule of the invention include introducing into a subject a labelled antibody directed against amino acids 62-100 of zyxin.

In addition, methods of the invention for differential diagnosis of healthy subjects, subjects having a non-malignant disease of the large intestine/colon, subjects having a precancerous colorectal lesion, subjects having a localized cancer of the large intestine/colon, subjects having a metastasised cancer of the large intestine/colon and/or subjects having an acute or chronic inflammation of colorectal tissue, described herein may be combined with other diagnostic methods to improve the outcome of the differential diagnosis. Other diagnostic methods are known to those skilled in the art.

As shown in the example above (for the differentiation of colorectal cancer from benign large intestine/colon hyperplasia), methods of the invention can also be used for the differential diagnosis of healthy subjects, subjects having a precancerous colorectal lesions, subjects having a non-malignant disease of the large intestine/colon, subjects having a localized cancer of the large intestine/colon, subjects having metastasised cancer of the large intestine/colon, and/or subjects having acute or chronic inflammation of the large intestine/colon.

### Biological Samples of the Invention

In more than one embodiment of the invention, biomolecules of the invention can be detected in blood, serum, plasma, urine, semen, seminal fluid, seminal plasma, pre-ejaculatory fluid (Cowper's fluid), nipple aspirate, vaginal fluid, excreta, tears, saliva, sweat, biopsy, ascites, cerebrospinal fluid, lymph, or tissue extract (biopsy) samples. Preferably, biological samples used to detect the biomolecules of the invention are of urine, blood, serum, plasma and excreta. Amino acids 62-100 of zyxin has been shown (below) to be present in blood and in plasma, and, as a molecule of less than 6000 mW, is also likely present in urine.

Furthermore, biological samples used for methods of the invention can be isolated from subjects of mammalian origin, preferably of primate origin. Even more preferred are subjects of human origin.

A subject of the invention that is said to have colorectal cancer possesses morphological, biochemical, and functional alterations of their colorectal tissue such that the tissue can be characterised as a malignant neoplasm. The stage to which a colorectal cancer has progressed can be determined using known methods currently available to those skilled in the art [e.g. Union Internationale Conte Cancer (UICC) system or American Joint Committee on Cancer (AJC)]. Currently, the most widely used method for determining the extent of malignancy of a colorectal neoplasm is the Gleason Grading system. Gleason grading is based exclusively on the architectural pattern of the glands of a colorectal neoplasm, wherein the ability of neoplastic cells to structure themselves into glands resembling those of the normal large intestine/colon is evaluated using a scale of 1 to 5. For example, neoplastic cells that are able to architecturally structure themselves such that they resemble normal large intestine/colon gland structure are graded 1-2, whereas neoplastic cells that are unable to do so are graded 4-5. As known to those skilled in the art, a colorectal neoplasm whose tumour structure is nearly normal will tend to behave, biologically, as normal tissue and therefore it is unlikely that it will be aggressively malignant.

A subject of the invention that is said to have a non-malignant disease of the large intestine/colon possesses morphological and/or biochemical alterations of their colorectal tissue but does not exhibit malignant neoplastic properties known to those skilled in the art. Such diseases include, but are not limited to, inflammatory and proliferative lesions, as well as benign disorders of the large intestine/colon. Within the context of the invention, whereas inflammatory diseases encompass Inflammatory Bowel Diseases including but not limited to Crohn disease, Ulcerative colitis, and proliferative lesions include benign large intestine/colon hyperplasia (BPH).

### Biologically Active Surfaces

Biologically active surfaces of the invention include, but are not limited to, surfaces that contain adsorbents with anion exchange properties (adsorbents that are positively charged), cation exchange properties (adsorbents that are negatively charged), hydrophobic properties, reverse phase chemistry, groups such as nitriloacetic acid that immobilize metal ions such as nickel, gallium, copper, or zinc (metal affinity interaction), or biomolecules such as proteins, antibodies, nucleic acids, or protein binding sequences, covalently bound to the surface via carbonyl diimidazole moieties or epoxy groups (specific affinity interaction). These surfaces may be located on matrices like polysaccharides such as sepharose, e.g. anion exchange surfaces or hydrophobic interaction surfaces, or solid metals, e.g. antibodies coupled to magnetic beads or a metal surface. Surfaces may also include gold-plated surfaces such as those used for Biacore Sensor Chip technology. Other surfaces known to those skilled in the art are also included within the scope of the invention.

Biologically active surfaces are able to adsorb biomolecules like nucleotides, nucleic acids, oligonucleotides, polynucleotides, amino acids, polypeptides, proteins, monoclonal and/or polyclonal antibodies, steroids, sugars, carbohydrates fatty acids, lipids, hormones, and combinations thereof (e.g., glycoproteins, ribonucleoproteins, lipoproteins).

In another embodiment, devices that use biologically active surfaces to selectively adsorb biomolecules may be chromatography columns for Fast Protein Liquid Chromatography (FPLC) and High Pressure Liquid Chromatography (HPLC), where the matrix, e.g. a polysaccharide, carrying the biologically active surface, is filled into vessels (usually referred to as “columns”) made of glass, steel, or synthetic materials like polyetheretherketone (PEEK).

In yet another embodiment, devices that use biologically active surfaces to selectively adsorb biomolecules may be metal strips carrying thin layers of a biologically active surface on one or more spots of the strip surface to be used as probes for gas phase ion spectrometry analysis, for example the Sax2 of Q10 ProteinChip® array for (Ciphergen Biosystems, Inc.) for SELDI analysis.

### Generation of Mass Profiles

In one embodiment, a mass profile of a biological sample may be generated using an array-based assay in which the biomolecules of a given sample are bound by biochemical or affinity interactions to an adsorbent present on a biologically active surface located on a solid platform (“chip”). After the biomolecules have bound to the adsorbent, they are co-crystallized with an energy absorbing molecule and subsequently detected using gas phase ion spectrometry. This includes, e.g., mass spectrometers, ion mobility spectrometers, or total ion current measuring devices. Quantity and characteristics of a biomolecule can be determined using gas phase ion spectrometry. Other substances in addition to a biomolecule of interest can also be detected by gas phase ion spectrometry.

In one embodiment, a mass spectrometer can be used to detect a biomolecule(s) on a chip. In a typical mass spectrometer, a chip with a bound biomolecule(s) co-crystallized with an energy absorbing molecule is introduced into an inlet system of the mass spectrometer. The energy absorbing molecule:biomolecule crystals are then ionized by an ionization source, such as a laser. The ions generated are then collected by an ion optic assembly, and then a mass analyzer disperses and analyzes the passing ions. The ions exiting the mass analyzer are then detected by an ion detector. The ion detector then translates the information into mass-to-charge ratios. Detection of the presence of a biomolecule(s) or other substances will typically involve detection of signal intensity. This, in turn, can reflect the quantity and character of a biomolecule bound to the probe.

In another embodiment, the mass profile of a sample may be generated using a liquid-chromatography (LC)-based assay in which biomolecule(s) of a given sample are bound by biochemical or affinity interactions to an adsorbent located in a vessel made of glass, steel, or synthetic material; known to those skilled in the art as a chromatographic column. The biomolecule(s) are eluted from the biologically active adsorbent surface by washing the vessel with appropriate solutions known to those skilled in the art. Such solutions include but are not limited to, buffers, e.g. Tris (hydroxymethyl) aminomethane hydrochloride (TRIS-HCl), buffers containing salt, e.g., sodium chloride (NaCl), or organic solvents, e.g., acetonitrile. Mass profiles of these biomolecules are generated by application of the eluting biomolecules of the sample by direct connection via an electrospray device to a mass spectrometer (LC/ESI-MS).

MALDI is a well known technique and is described in Brummell et al., Science 264: 399-402 (1994), which is hereby incorporated by reference. In MALDI, a sample is partially purified to obtain a fraction that comprises a biomolecule by employing such separation methods as: two-dimensional gel electrophoresis (2D-gel) or high performance liquid chromatography (HPLC). Specifically, sample(s) and matrix with a positive charge are mixed together and flashed with a laser. The matrix becomes ionized (MH+) with an extra proton and then the proton is transferred to the sample to create a positively charged sample(s). The charged sample(s) is then run through a detector where the smaller ions reach the detector first and then the larger ions. This is the time of flight (TOF), and the mass to charge ratio (M/Z) is proportional to the square of the drift time.

In another embodiment, surface-enhanced laser desorption/ionisation mass spectrometry (SELDI) can be used to detect a biomolecule, which can be PSP94 and/or PSA, and uses a substrate comprising adsorbents to capture biomolecules, which can then be directly desorbed and ionised from the substrate surface during mass spectrometry. Since the substrate surface in SELDI captures biomolecules, a sample need not be partially purified as in MALDI. However, depending on the complexity of a sample and the type of adsorbents used, it may be desirable to prepare a sample to reduce its complexity prior to SELDI analysis. The SELDI is described, inter alia, in U.S. Pat. Nos. 5,719,060, 6,225,047, 6,579,719, and 6,818,411, which are hereby incorporated by reference.

Conditions that promote binding of a biomolecule(s) to an adsorbent are known to those skilled in the art and ordinarily include parameters such as pH, the concentration of salt, organic solvent, or other competitors for binding of the biomolecule to the adsorbent.

### Detection of Biomolecules

In another aspect of the invention, amino acids 62-100 of zyxin can be detected using other methods known to those skilled in the art. For example an in vitro binding assay can be used to detect a biomolecule of the invention within a biological sample of a given subject. A given biomolecule of the invention can be detected within a biological sample by contacting the biological sample from a given subject with specific binding molecule(s) under conditions conducive for an interaction between the given binding molecule(s) and amino acids 62-100 of zyxin. Binding molecules include, but are not limited to, nucleic acids, nucleotides, oligonucleotides, polynucleotides, amino acids, peptides, polypeptides, proteins, monoclonal and/or polyclonal antibodies, antigens, sugars, carbohydrates, fatty acids, lipids, steroids, or combinations thereof. (e.g. glycoproteins, ribonucleoproteins, lipoproteins), compounds or synthetic molecules.

Preferably, binding molecules are antibodies specific for amino acids 62-100 of zyxin. Biomolecules that can be detected using the above-mentioned binding molecules include, but are not limited to, molecules comprising nucleic acids, nucleotides, oligonucleotides, polynucleotides, amino acids, peptides, polypeptides, proteins, monoclonal and/or polyclonal antibodies, antigens, sugars, carbohydrates, fatty acids, lipids, steroids, and combinations thereof (e.g., glycoproteins, ribonucleoproteins, lipoproteins). Preferably, biomolecules that can be detected using the above-mentioned binding molecules include, nucleic acids, nucleotides, oligonucleotides, polynucleotides, amino acids, peptides, polypeptides, proteins, monoclonal and/or polyclonal antibodies. Even more preferred are binding molecules that are amino acids, peptides, polypeptides, proteins, monoclonal and/or polyclonal antibodies.

### Antibodies

With respect to protein-based testing, antibodies can be generated to the biomarkers using standard immunological techniques, fusion proteins or synthetic peptides as described herein. Monoclonal antibodies can also be produced using now conventional techniques such as those described in Waldmann (1991) and Harlow and Lane (1988). It will also be appreciated that antibody fragments, e.g., Fab′ fragments, can be similarly employed. Immunoassays, for example ELISAs, in which a test sample is contacted with antibody and binding to the biomarker detected, can provide a quick and efficient method of determining the presence and quantity of the biomarker. For example, antibodies can be used to test effects of pharmaceuticals in subjects enrolled in clinical trials.

Thus, the present invention also provides polyclonal and/or monoclonal antibodies and fragments thereof, and immunologic binding equivalents thereof, which are capable of specifically binding to biomarkers of the invention and fragments thereof. The term “antibody” is used both to refer to a homogeneous molecular entity, or a mixture such as a serum product made up of a plurality of different molecular entities. Polypeptides may be prepared synthetically in a peptide synthesizer and coupled to a carrier molecule (e.g., keyhole limpet hemocyanin) and injected over several months into a host mammal. A host's sera can be tested for immunoreactivity to a subject polypeptide or fragment. Monoclonal antibodies may be made by injecting mice with the protein polypeptides, fusion proteins, or fragments thereof. Monoclonal antibodies are screened by ELISA and tested for specific immunoreactivity with subject biomarkers or fragments thereof (Harlow & Lane, 1988). These antibodies are useful in assays as well as a therapeutic drug.

Once a sufficient quantity of desired polypeptide has been obtained, it may be used for various purposes. A typical use is the production of antibodies specific for binding. These antibodies may be either polyclonal or monoclonal, and may be produced by in vitro or in vivo techniques well known in the art. For production of polyclonal antibodies, an appropriate target immune system, typically mouse or rabbit, is selected. Substantially purified antigen is presented to the immune system in a fashion determined by methods appropriate for the animal and by other parameters well known to immunologists. Typical routes for injection are in footpads, intramuscularly, intraperitoneally, or intradermally. Of course, other species may be substituted for mouse or rabbit. Polyclonal antibodies are then purified using techniques known in the art, adjusted for the desired specificity.

An immunological response is usually assayed with an immunoassay. Normally, such immunoassays involve some purification of a source of antigen, for example, that produced by the same cells and in the same fashion as the antigen. A variety of immunoassay methods are well known in the art, such as in Harlow and Lane (1988) or Goding (1996).

Monoclonal antibodies with affinities of 10⁸ M⁻¹ or preferably 10⁹ to 10¹⁰ M⁻¹ or stronger will typically be made by standard procedures as described in Harlow and Lane (1988) or Goding (1996). Briefly, appropriate animals will be selected and the desired immunization protocol followed. After the appropriate period of time, the spleens of such animals are excised and individual spleen cells fused, typically, to immortalized myeloma cells under appropriate selection conditions. Thereafter, the cells are clonally separated and the supernatants of each clone tested for their production of an appropriate antibody specific for the desired region of the antigen.

Other suitable techniques involve in vitro exposure of lymphocytes to antigenic biomarkers, or alternatively, to selection of libraries of antibodies in phage or similar vectors (Huse et al., 1989). Polypeptides and antibodies of the present invention may be used with or without modification. Polypeptides and antibodies can be labelled by joining, either covalently or non-covalently, a substance, which provides for a detectable signal. A wide variety of labels and conjugation techniques are known and are reported extensively in both the scientific and patent literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced (see U.S. Pat. No. 4,816,567).

### Generation of Monoclonal Antibodies Specific for a Biomarker

Monoclonal antibodies can be generated according to various methods known to those skilled in the art. For example, any technique that provides for the production of antibody molecules by continuous cell lines in culture may be used. These include but are not limited to the hybridoma technique originally developed by Kohler and Milstein (1975), as well as the trioma technique, the human B-cell hybridoma technique (Kozbor et al., 1983; Cote et al., 1983), and the EBV-hybridoma technique to produce human monoclonal antibodies (Cole et al., 1985). In fact, techniques developed for producing “chimeric antibodies” (Morrison et al., 1984; Neuberger et al., 1984; Takeda et al., 1985) by splicing genes from a mouse antibody molecule specific for a given biomarker of the invention together with genes from a human antibody molecule of appropriate biological activity can be used. Such human or humanized chimeric antibodies are preferred for use in therapy of human diseases or disorders since the human or humanized antibodies are much less likely than xenogeneic antibodies to induce an immune response, in particular an allergic response.

The following example of monoclonal antibody production is meant for clarity and is not intended to limit the scope of the invention. One method to producing antibodies of the invention is by inoculating a host mammal with an immunogen comprising the intact subject biomarker or its peptides (wild or mutant). The host mammal may be any mammal and is preferably a host mammal such as a mouse, rat, rabbit, guinea pig or hamster and is most preferably a mouse. By inoculating the host mammal it is possible to elicit the generation of antibodies directed towards the immunogen introduced into the host mammal. Several inoculations may be required to elicit an immune response.

To determine if the host mammal has developed antibodies directed towards the immunogen, serum samples are taken from the host mammal and screened for the desired antibodies. This can be accomplished by techniques known in the art such as radioimmunoassay, ELISA (enzyme-linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immnunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled.

Once antibody generation is established in the host mammal, it is selected for hybridoma production. The spleen is removed and a single cell suspension is prepared as described by Harlow and Lane (1988). Cell fusions are performed essentially as described by Kohler and Milstein (1975). Briefly, P3.65.3 myeloma cells (American Type Culture Collection, Rockville, Md.) are fused with immune spleen cells using polyethylene glycol as described by Harlow and Lane (1988). Cells are plated at a density of 2×10⁵ cells/well in 96 well tissue culture plates. Individual wells are examined for growth and the supernatants of wells with growth are tested for the presence of subject biomarker specific antibodies by ELISA or RIA using wild type or mutant target protein. Cells in positive wells are expanded and subcloned to establish and confirm monoclonality. Clones with the desired specificities are expanded and grown as ascites in mice or in a hollow fiber system to produce sufficient quantities of antibody for characterization and assay development.

### Sandwich Assay for the Biomarker

Sandwich assays for detecting amino acids 62-100 of zyxin can be used as a diagnostic tool for a diagnosis of a subject as being healthy, having a non-malignant disease of the large intestine/colon, having a precancerous colorectal lesion, having a localized cancer of the large intestine/colon, or a metastasised cancer of the large intestine/colon, or having an acute or a chronic inflammation of colorectal tissue. In the context of the invention, sandwich assays consist of attaching a monoclonal antibody to a solid surface such as a plate, tube, bead, or particle, wherein the antibody is preferably attached to the well surface of a 96-well microtitre plate. A pre-determined volume of sample (e.g., serum, urine, tissue cytosol) containing the subject biomarker is added to the solid phase antibody, and the sample is incubated for a period of time at a pre-determined temperature conducive for the specific binding of the subject markers within the given sample to the solid phase antibody. Following, the sample fluid is discarded and the solid phase is washed with buffer to remove any unbound material. One hundred μl of a second monoclonal antibody (to a different determinant on the subject biomarker) is added to the solid phase. This antibody is labelled with a detector molecule or atom (e.g., enzyme, fluorophore, chromophore, or ¹²⁵I), and the solid phase with the second antibody is incubated for two hrs at room temperature. The second antibody is decanted and the solid phase is washed with buffer to remove unbound material.

The amount of bound label, which is proportional to the amount of subject biomarker present in the sample is quantitated.

### Competition Assay for the Biomarker

Competition assays for the detection of amino acids 62-100 of zyxin can be used as a diagnostic tool for the diagnosis of a subject as being healthy, having a non-malignant disease of the large intestine/colon, having a precancerous colorectal lesion, having a localized cancer of the large intestine/colon, or a metastasised cancer of the large intestine/colon, or having an acute or a chronic inflammation of colorectal tissue. In the context of the invention, competition assays consist of attaching a monoclonal antibody to a solid surface such as a plate, tube, bead, or particle, wherein the antibody is preferably attached to the well surface of a 96-well microtitre plate. A pre-determined volume of sample (e.g., serum, urine, tissue cytosol) containing the subject biomarker is added to the solid phase antibody in the presence of labelled SEQ ID NO:1, wherein the label is enzyme, fluorophore, chromophore, ¹²⁵I, etc., and the sample is incubated for a period of time at a pre-determined temperature conducive for the specific binding of the subject markers within the given sample to the solid phase antibody. Following, the sample fluid is discarded and the solid phase is washed with buffer to remove any unbound material. SEQ ID NO:1 (amino acids of 62-100 of zyxin) is labelled with a detector molecule or atom (e.g., enzyme, fluorophore, chromophore, ¹²⁵I, biotin, etc). Thereby, unlabeled zyxin in the specimen competes for the labelled zyxin fragment (SEQ ID NO:1), and the detector molecule decreases with increasing concentration of zyxin in the specimen.

### Kits

In yet another aspect, the invention provides kits using the methods of the invention as described in another section for the differential diagnosis of a colorectal cancer or a non-malignant disease of the large intestine/colon, wherein the kits are used to detect amino acids 62-100 of zyxin.

The methods used to detect amino acids 62-100 of zyxin can also be used to determine whether a subject is at risk of developing a colorectal cancer or has developed a colorectal cancer. Such methods may also be employed in the form of a diagnostic kit comprising a binding molecule specific to amino acids 62-100 of zyxin, solutions and materials necessary for the detection of a biomolecule of the invention, and instructions to use the kit based on the above-mentioned methods.

For example, kits can be used to detect amino acids 62-100 of zyxin. Kits of the invention have many applications. For example, kits can be used to differentiate if a subject is healthy, having a non-malignant disease of the large intestine/colon, or a colorectal cancer, thus aiding the diagnosis of a colorectal cancer and/or a non-malignant disease of the large intestine/colon. Moreover, the kits can be used to differentiate if a subject healthy, having a non-malignant disease of the large intestine/colon, having a precancerous colorectal lesion, having a localized cancer of the large intestine/colon, having a metastasised cancer of the large intestine/colon, or having an acute or a chronic inflammation of the large intestine/colon.

In one embodiment, a kit comprises instructions on how to use the kit, an adsorbent on a biologically active surface, wherein the adsorbent is suitable for binding one or more biomolecules of the invention, a denaturation solution for the pre-treatment of a sample, a binding solution, and one or more washing solution(s) or instructions for making a denaturation solution, binding solution, or washing solution(s), wherein the combination allows for the detection of a biomolecule using gas phase ion spectrometry. Such kits can be prepared from the materials described in other previously detailed sections (e.g., denaturation buffer, binding buffer, adsorbents, washing solution(s), etc.).

In some embodiments, a kit may comprise a first substrate comprising an adsorbent thereon (e.g., a particle functionalised with an adsorbent) and a second substrate onto which the first substrate can be positioned to form a probe, which is removably insertable into a gas phase ion spectrometer. In other embodiments, a kit may comprise a single substrate, which is in the form of a removably insertable probe with adsorbents on the substrate.

In another embodiment, a kit comprises a binding molecule or panel of binding molecules that specifically binds to amino acids 62-100 of zyxin a detection reagent, appropriate solutions and instructions on how to use the kit. Such kits can be prepared from the materials described above, and other materials known to those skilled in the art. A binding molecule used within such a kit may include, but is not limited to, nucleic acids, nucleotides, oligonucleotides, polynucleotides, amino acids, peptides, polypeptides, proteins, monoclonal and/or polyclonal antibodies, sugars, carbohydrates, fatty acids, lipids, steroids, hormones, or a combination thereof (e.g. glycoproteins, ribonucleoproteins, lipoproteins), compounds or synthetic molecules). Preferably, a binding molecule used in said kit is a nucleic acid, nucleotide, oligonucleotide, polynucleotide, amino acid, peptide, polypeptide, and protein, monoclonal and/or polyclonal antibody.

In the embodiment, a kit may optionally further comprise a standard or control biomolecule so that the biomolecule detected within the biological sample can be compared with said standard to determine if the test amount of a marker detected in a sample is a diagnostic amount consistent with a diagnosis of a non-malignant disease of the large intestine/colon, a precancerous colorectal lesion, localized cancer of the large intestine/colon, metastasised cancer of the large intestine/colon, acute or a chronic inflammation of the large intestine/colon. Likewise a biological sample can be compared with said standard to determine if the test amount of a marker detected is said sample is a diagnostic amount consistent with a diagnosis as healthy.

### Composition, Formulation, and Administration of Pharmaceutical Compositions

Differential expression of a biomolecule in samples from healthy subjects, subjects having a non-malignant disease of the large intestine/colon, and subjects having colorectal cancer allows for differential diagnosis of a colorectal cancer or a non-malignant disease of the large intestine/colon within a given subject. Knowledge of the association of these biomolecules with colorectal cancer and benign large intestine/colon disease can be used, for example, to treat patients with the biomolecule, an antibody specific to the biomolecule, or an antagonist of the biomolecule. In order to treat colorectal cancer, the biomolecules can be prepared in specific pharmaceutical compositions and/or formulations that allow for the most efficient and effective delivery of the therapy.

Pharmaceutical compositions of the present invention may be manufactured by known methods, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations, which can be used pharmaceutically. Proper formulation is dependent upon the route of administration chosen.

For injection, agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol, or cellulose preparations such as, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, compositions may take the form of tablets or lozenges formulated in conventional manner.

For administration by inhalation, compounds for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges (e.g. gelatin) for use in an inhaler or insufflator may be formulated containing a powder mix of the compound and a suitable powder base such as lactose or starch.

Compounds may be formulated for parenteral administration by injection, e.g., by bolus injection or continuous infusion. Formulations for injection may be presented in unit dosage form, e.g., in ampoules or in multidose containers, with an added preservative. Compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

Compounds may also be formulated in rectal compositions such as suppositories or retention enemas, e.g., containing conventional suppository bases such as cocoa butter or other glycerides.

In addition to the formulations described previously, compounds may also be formulated as a depot preparation. Such long acting formulations may be administered by implantation (for example subcutaneously or intramuscularly) or by intramuscular injection. Thus, for example, compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system comprising benzyl alcohol, a nonpolar surfactant, a water-miscible organic polymer, and an aqueous phase. Naturally, proportions of a co-solvent system may be varied considerably without destroying its solubility and toxicity characteristics. Furthermore, identity of the co-solvent components may be varied.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, compounds may be delivered using a sustained-release system, such as semipermeable matrices of solid hydrophobic polymers containing therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of therapeutic reagent, additional strategies for protein stabilization may be employed.

Pharmaceutical compositions may also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Compounds of the invention may also be provided as salts with pharmaceutically compatible counterions. Pharmaceutically compatible salts may be formed with many acids, including but, not limited to, hydrochloric, sulfuric, acetic, lactic, tartaric, malic, succinic, etc. Salts tend to be more soluble in aqueous or other protonic solvents than are the corresponding free base forms.

Suitable routes of administration may, for example, include oral, rectal, transmucosal, transdermal, or intestinal administration; or parenteral delivery, including intramuscular, subcutaneous, intramedullary injections, as well as intrathecal, direct intraventricular, intravenous, intraperitoneal, intranasal, or intraocular injections.

Alternately, one may administer a compound in a local rather than systemic manner, for example, via injection of the compound directly into an affected area, often in a depot or sustained release formulation.

Furthermore, one may administer a drug in a targeted drug delivery system, for example, in a liposome coated with an antibody specific for affected cells. Liposomes can be targeted to and taken up selectively by cells.

Pharmaceutical compositions generally are administered in an amount effective for treatment or prophylaxis of a specific indication or indications. It is appreciated that optimum dosage will be determined by standard methods for each treatment modality and indication, taking into account the indication, its severity, route of administration, complicating conditions and the like. In therapy or as a prophylactic, an active agent may be administered to an individual as an injectable composition, for example, as a sterile aqueous dispersion, preferably isotonic. A therapeutically effective dose further refers to that amount of the compound sufficient to result in amelioration of symptoms associated with such disorders. Techniques for formulation and administration of the compounds of the instant application may be found in ‘Remington's Pharmaceutical Sciences,’ Mack Publishing Co., Easton, Pa., latest edition. For administration to mammals, and particularly humans, it is expected that the daily dosage level of the active agent will be from 0.001 mg/kg to 10 mg/kg, typically around 0.01 mg/kg. The physician in any event will determine the actual dosage, which will be most suitable for an individual and will vary with the age, weight and response of the particular individual. The above dosages are exemplary of the average case. There can, of course, be individual instances where higher or lower dosage ranges are merited, and such are within the scope of this invention.

Compounds of the invention may be particularly useful in animal disorders (veterinarian indications), and particularly mammals.

The invention further includes diagnostic and pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

The present invention is further illustrated by the following examples, which should not be construed as limiting in any way. The contents of all cited references (including literature references, issued patents, published patent applications), as cited throughout this application, are hereby expressly incorporated by reference. The practice of the present invention will employ, unless otherwise indicated, conventional techniques of cell biology, cell culture, molecular biology, transgenic biology, microbiology, recombinant DNA, and immunology, which are known to those skilled in the art. Such techniques are explained fully in the literature.

### EXAMPLES

### Example 1

### Sample Collection for Colon Cancer Evaluation

Serum samples were obtained from ETSI (European Tumour Sample Institute, Hennigsdorf, Germany), which included three different groups of subjects from patients recruited through the department of Gastroenetrology and Surgery at the Universities of Erlangen and Magdeburg (both in Germany). Group A comprised sera drawn from 68 colorectal cancer patients that had been collected directly before surgery. Diagnosis was made based on endoscopy, ultrasonic testing, and/or other means of colorectal cancer detection, and was confirmed by post-surgical histological evaluation.

Group B consisted of sera drawn from 45 patients with non-malignant (“benign”) disease symptoms of the large intestine (for example, adenoma, inflammation, diverticulitis). Sera were collected following colorectal endoscopy to confirm the absence of colorectal cancer.

Group C consisted of sera drawn from 23 healthy patients who were not suffering from severe disease at the time of sample collection.

TABLE 1
Summary of the distribution of samples for the
discovery of biomarkers for colorectal cancer.

Gender

| | Male | | | Female | | |
|---|---|---|---|---|---|---|
| Site | E | MD | Total | E | MD | Total |
| CRCa | 5 | 31 | 36 | 3 | 29 | 32 |
| Benign | 0 | 18 | 18 | 0 | 27 | 27 |
| Healthy | 0 | 9 | 9 | 0 | 14 | 14 |

CRCa—colorectal cancer, MD—Magdeburg, E—Erlangen

### Example 2

### Sample Preparation

136 serum samples (100 μL aliquots) stored at −80° C. were thawed at room temperature and placed on ice immediately. 15 μL of each serum sample was mixed with 60 μL at Lysis Solution E (7M Urea, 2M Thiourea, 4% CHAPS, 1% DTT and 2% Ampholine) in a set of 1.5 mL microcentrifuge tubes and samples were incubated on ice for 15 min. After incubation, 675 μL of Binding Buffer SAX2 (0.1M Tris HC1 pH8.5) was added to each of the samples. All samples were then placed on ice.

### Example 3

### Sample Analysis by Mass Spectrometry

Serum samples were randomly applied to Q10 ProteinChip® array surfaces that consist of cationic quaternary amines groups. Such array surfaces are selective for molecules that are negatively charged. Pooled serum (quality control) and PBS (negative control) were also applied to each array to control for inter-array bias. All samples were applied in duplicate. Following in-house standard operating procedures, samples were processed directly on the array surfaces and subsequently assayed using a PCS4000 SELDI-TOF MS over a mass range of 0 to 30,000 m/z and the energy absorbing molecule sinapinic acid (SPA). The spectra generated for each applied sample were normalized for total ion current using the Normalize Spectra functionality of CiphergenExpress® version 3.0 over a mass range of 1,500 to 30,000 m/z. The mean and standard deviation for the distribution of normalization factors applied to spectra (excluding those generated from quality assurance spots) were calculated and those spectra with a normalization factor of more than two standard deviations from the mean were discarded. Based on the above criteria, a select number of markers were identified by m/z ratio. The proteins/peptides associated with these m/z points were analyzed for viability as markers for colorectal cancer. The most promising single marker, i.e., the peak at 4949 Da, was identified as the 62-110 amino acid fragment of the protein zyxin.

### Example 4

### Identification of a Fragment of Zyxin, the Peak at 4949Da

In SELDI, the peak was detected at the molecular mass of [M+H]1+=4963Da (+−0.5%)(average mass). MALDI-TOF measurements of purified fractions determined the molecular mass of [M+H]1+=4949Da(+−0.1%)(average mass). Peak 4949Da was purified from healthy blood donor serum (XHMX0008292 and XHMX0008294).3500; 11 Serum (175011 respectively) was resuspended in 2500111 denaturing buffer (7M urea, 2M thiourea, 1% DTT and 0.02% Triton X®-100) and incubated on ice for 10 min. The chromatographic steps were performed (i) at 4° C. by using the Akta® system (Amersham Biosciences, Uppsala, Sweden) or (ii) at 10° C. by using the Vision Workstation (Applied Biosystems, Foster City, Calif., USA).

The size exclusion chromatography of the diluted serum was performed on a Sephacryl® S-300 HiPrep® 16160 and Sephacryl® S-100 HiPrep® 16160 column (Amersham Biosciences) connected in a row with 0.1 M Tris, pH8.5, 0.25 M urea, 0.08% DTT, 0.02% Triton X®-100 and 250 mM NaCl.

All fractions were analyzed by MALDI-TOF. 20 μl of a fraction was concentrated and desalted using ZipTip_{μ-C18} (Millipore, Billerica, Mass., USA) according to the user manual. ZipTips® were washed with 50% acetonitrile, 0.1% TFA and equilibrated with 0.1% TFA. 0.1% TFA was used as washing solution. Elution was performed with 1.5 μl matrix solution (20 mg/ml sinapinic acid in 50% acetonitrile, 0.3% TFA) directly onto the MALDI target. Measurements were performed on a Voyager-DE STR MALDI-TOF (Applied Biosystems) mass spectrometer. Spectra of the following mass ranges were measured: 5805000Da (reflector mode, 20 kV accelerating voltage, delay time 200 nsec, low mass gate 580 Da), 4000 25000Da (linear mode, 25 kV accelerating voltage, delay time 600 nsec, low mass gate 4000Da), 20000 100000Da (linear mode, 25 kV accelerating voltage, delay time 850 nsec, low mass gate 5000Da). Per spectra, 10 single measurements of 100-150 shots were accumulated. External calibration was performed using a Peptide/Protein mix from Laserbio (Sophia-Antipolis Cedex, France).

The peak at 4949Da eluted at the appropriate molecular weight. The most intense fractions (according to MALDI measurement) were combined and precipitated (TCA-DOC precipitation), by adding 1/100 vol. of 2% DOC (deoxycholate) to one volume of protein solution, vortexed and incubated for 30 min at 4° C. Subsequently 1110 vol. of TCA was added, the sample was vortexed and incubated on ice for at least 15 min. Afterwards centrifugation was performed at 15000 g for 10 min at 4° C. The pellet was dried by inverting the tube. Pellet was washed twice with one volume cold acetone (vortex and repellet sample 5 min at full speed between washes). The sample was dried in a speed vac and resuspended in a minimal volume of sample buffer (100 mM NaAc, pH4.5, 0.25 M urea, 0.08% DTT).

The pooled sample was chromatographed on a Mono S™ HR5/5 (Amersham Biosciences) column (bed volume, 1 ml) with 100 mM NaAc, pH4.5, 0.25 M urea, 0.08% DTT and a gradient from 0 M to 2 M NaCl over 20 ml. All fractions were analyzed by MALDI-TOF as described above.

The flow through fractions were combined and precipitated (TCA-DOC precipitation) as described above.

The pooled sample was dissolved in running buffer and chromatography was performed on a Mono Q™ HR 5/5 column (Amersham Biosciences) with 0.1 M Tris, pH8.5, 0.25 M urea, 0.08% DTT and a linear NaCl gradient from 0 to 1 M over 20 ml for elution of the proteins. All fractions were analyzed by MALDI-TOF as described above. The peak 4949Da eluted at about 0.3M NaCl.

The fractions containing the peak at 4949Da were redissolved in 500 μl 2.5% acetonitrile, 0.1% TFA and applied to a reversed phase column. RP-HPLC was performed on a Vision Workstation (AppliedBiosystems) at 10° C. using a 100×2 mmC8 Column (Prontosil 300-5-C8 SH 511 m, Bischoff, Leonberg, Germany). Eluent A was 0.1% TFA in 95% H₂O, 5% acetonitrile; buffer B was 0.085% TFA in 95% acetonitrile, 5% H₂O. The gradient applied was linear from 0% B to 20% B in 3 min; 20% B to 45% B in 30 min and 45% B to 100% B in 3 min. All fractions of reversed-phase chromatography were dried in a vacuum concentrator and redissolved in 5 μl 50% acetonitrile, 0.1% TFA. 0.7 μl redissolved sample was mixed with 0.7 μl matrix (20 mg/ml sinapinic acid in 50% acetonitrile, 0.3% TFA) and 1 μl was applied onto the MALDI target. Measurements were performed on a Voyager-DE STR MALDI-TOF (Applied Biosystems) mass spectrometer as described above. The peak 4949Da eluted at about 40% B.

The mass 4949Da was analyzed with MALDI Post-Source Decay (PSD). Measurements were performed on a Voyager-DE STR MALDI-TOF (Applied Biosystems) mass spectrometer using the PSD modus. 10single spectra were measured and automatically stitched to the complete PSD spectrum. For each single spectrum, 3-5 spectra with 1000 shots were accumulated. A database search was performed using the program MS-Tag (Protein Prospector). The database search ranked Zyxin (SwissProt Q15942) as match number one.

The remaining fraction containing the peak 4949Da was diluted with 30 μl 0.1% TFA and then processed with ZipTip®_{μ-C18} (Millipore). Elution was performed with 2.5 μl 50% acetonitrile, 0.1% formic acid (FA). The eluate was analyzed by nano-electrospray MS/MS using a Q-TOF Micro (Micromass, Manchester, UK). ESI-MS/MS measurement was performed for m/z [M+4Ht+=1236.89. The molecular mass determined with ESI-MS was [M]=4943.52Da (+−0.01%) (monoisotopic mass). The spectra were interpreted manually. Detected sequence information was matched with the sequence of zyxin which was result of the MALDI PSD measurement. The main signals could be matched to the mentioned sequence. MALDI PSD data and ESI MS/MS data are complementary and the peptide was identified as amino acids 62-110 of zyxin (SwissProt Q15942; calculated monoisotopic molecular mass [M]=4943.41Da).

### Data Selected for Statistical Analysis

To eliminate the noise due to the variability in mass spectrometry readings, the data selected met the following validity criteria: (1) All data from mass spectrometry must be in duplicate for inclusion. (a) Reason: Variability of individual replicates was not as reliable as result from 2 replicates. (2) Positive control on the Q10 chip must have an intensity <30 to be included in the results (a) Reason: Pooled positive samples rarely exceeded an intensity of 30 (e.g., 5 pts of 133). The remaining data were analyzed by MedCalc Software 2008.

TABLE 2
Final number of samples

| Colorectal Cancer | Healthy Control | Benign |
|---|---|---|
| 37 | 10 | 30 |

### Results:

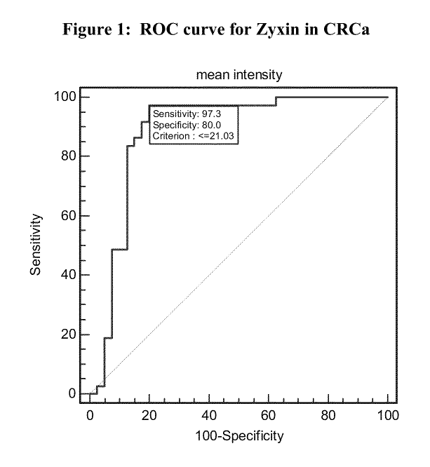

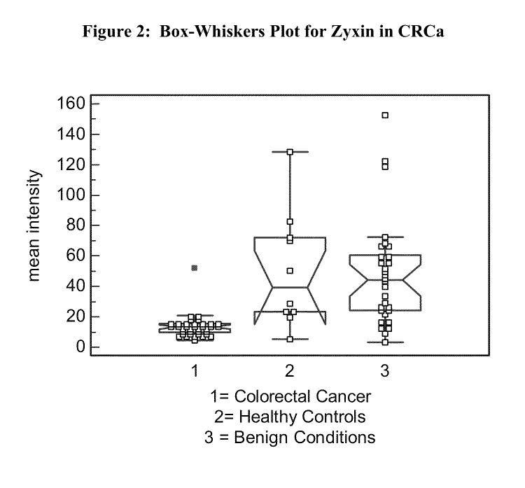

The performance of Zyxin at separating CRCa samples from Healthy/Benign is illustrated below. FIG. 1 illustrates a ROC curve with an area under the curve (AUC) of 0.884 (0.79 to 0.945) with a 97% sensitivity and 80% specificity. FIG. 2 shows the Box-Whisker Plot for the CRCa, healthy controls and Benign specimens. The median values are clearly separated between cancer and non cancer (P<0.0001, Kruskal-Wallis Test). Table 3 documents the median value for CRCa=13; non-cancer and health individuals median value was 44 and 39 respectively. The 95% confidence limits for the medians did not overlap between the cancer and non-cancer samples.

TABLE 3
Summary Statistics for CRCa (1), Healthy Controls (2), and Benign Samples (3)

| | number disease | | | | | | | | |
|---|---|---|---|---|---|---|---|---|---|
| | 1 | | | 2 | | | 3 | | |
| | N | Median | 95% CI | N | Median | 95% CI | N | Median | 95% CI |
| mean intensity | 37 | 13.320 | 10.620-14.853 | 10 | 39.355 | 17.950-87.685 | 30 | 44.160 | 25.727-57.833 |

Zyxin is thus significantly down regulated as compared to the healthy controls and benign samples and is a good candidate as a serum biomarker for colorectal cancer based on its ability to separate CRC patient samples from healthy controls or those with benign disease.

### Example 5

### Preparation of an Antibody to Amino Acids 62-100 of Zyxin

Preparation of Peptide-KLH:

Three overlapping 15 amino acid peptides (62-77, 75-100, 95-110) are synthesized representing 3 regions of the zyxin fragment (SEQ ID NO: 1) with a terminal cysteine. For 2 mg of each peptide, the terminal cysteine is covalently linked to 2 mg of an maleimide-activated carrier protein keyhole limpet hemocyanin (KLH). The peptide-KLH reaction mixture is run over a desalting column.

Preparation of Antibodies to Zyxin Peptide-KLH:

Two rabbits are selected for immunization and 4-5 mL of blood is drawn for preimmune sera. 1 mg of zyxin peptide-KLH is mixed in 0.5 mL of buffer and 0.5 mL of incomplete Freund's adjuvant and is injected on day zero. This injection is repeated 2 more times on day 14 and day 28. A test bleed is performed, where 4-5 mL of blood is collected for titer testing. If the rabbit has high titer of antibody, the animal is sacrificed and 60-80 mL of blood is collected. If titer is low, zyxin pepted-KLH is injected one more time; the rabbit is sacrificed fourteen days later and 60-80 mL of blood is collected.

Determination of Titration:

Each of the zyxin peptides is covalently coupled to maleimide-activated 96 well plates. Each well is washed 4 times with Wash Buffer [PBS (Phosphate buffer saline) with 0.05% Tween®] and then blocked with Assay Diluent (PBS containing 1% BSA (bovine serum albumin)). The blocking solution is removed and then incubated with serial dilutions of rabbit sera in blocking solution starting with 1:1000 dilution. Each well is washed 4 times with Wash Buffer followed by an incubation with a 1 ug/mL solution of anti-rabbit alkaline phosphatase. Each well is washed with Wash Buffer and then incubated with PNPP (p-Nitrophenyl Phosphate, Disodium Salt). The color development from the PNPP increases with increasing amounts of rabbit anti-sera against zyxin. The lowest concentration detectable over background binding of a non related peptide is the titer of the antibody.

Purification of Rabbit Polyclonal Antisera:

1 mL of rabbit antibody diluted into 1 mL 50 mM Acetate pH 5.0 is loaded onto a 1 mL protein G column. Wash with 10-15 column volumes of acetate buffer and then elute with 2-5 mL of 0.1 M glycine pH 2-3. Identify the protein peak by OD280 and then neutralize samples with 1M Phosphate. Purified IgG fraction is then run over a desalting column to exchange the buffer to PBS. The solution is then sterile filtered.

### Example 6

### Use of Zyxin for the Diagnosis of Colorectal Cancer

Manufacturing of Biotinylated-Zyxin Reagent:

One mL of zyxin peptide at 2 mg/mL is incubated with 400 uL of a 20 mM solution of Maleimide PEG₂-Biotin and incubated for greater than 2 hr at 2-8° C. After coupling, the biotinylated zyxin is run over a desalting column to remove excess biotin.

Quantitation of Zyxin by Competitive ELISA:

Two hundred μL of a 1 μg/mL affinity purified rabbit anti-zyxin antibodies in 50 mM Carbonate pH 9.0 are incubated overnight at 2-8° C. The anti-zyxin antibody coated plates are washed with Wash Buffer and blocked with 250 uL of Assay Buffer. The washed plates are incubated with 100 μL of human sera from patients, 100 μL of control sera (human sera known to be from colorectal cancer-free individuals) as well as calibrators and controls made from zyxin peptides. After a 1 hr incubation at room temperature, the plates are washed and then 100 uL of biotinylated zyxin is added to the plates. After a 1 hr incubation, the plates are washed again and 100 uL of 10 ug/ml solution of strepavidin-alkaline phosphatase is added to each well. The plate is washed PNPP is added to each well and then the colour is developed in 15 to 30 minutes. As unlabeled zyxin increases in the calibrators and controls, the amount of colour development decreases. Patients with colorectal cancer will have lower concentrations of zyxin (and thus higher color) as compared to colorectal-cancer free controls.

### Example 7

### Use of Amino Acids 62-100 of Zyxin for Treatment of Colorectal Cancer

Patients

diagnosed as having colorectal cancer using the diagnostic method of Example 6 are administered amino acids 62-100 of Zyxin. Specifically, 1 ml of zyxin peptide at 2 mg/ml in phosphate buffered saline is injected by IV injection into the bloodstream of the patient, once a day, for 21 days. A control group of patients having colorectal cancer, and similar levels of the 62-100 aa fragment of Zyxin (as determined using the diagnostic method of Example 6) are placebo treated with IV injections of saline only. Patient tumors are excised in traditional CRC surgery; tumors are significantly smaller than the controls.

1-33. (canceled)

34. A method for the diagnosis of a colorectal cancer in a subject comprising:
(a) obtaining a biological sample from the subject;
(b) detecting the quantity, presence or absence of SEQ ID NO:1 within said biological sample;
(c) classifying said subject as having or not having colorectal cancer based on the quantity, presence or absence of SEQ ID NO: 1 within the sample;
wherein the subject is classified as having colorectal cancer if the quantity of SEQ ID NO:1 detected is significantly below a quantity or range typically found in a similar biological sample from a healthy individual; and
initiating a treatment for colorectal cancer when the subject is classified as having colorectal cancer.

35. The method of claim 34, wherein the quantity, presence, or absence of the biomarker is detected in the biological sample obtained from the subject by antibody immunoassay.

36. The method of claim 35, wherein the method of antibody immunoassay is selected from the group consisting of EIA (enzyme immunoassay), RIA (radioimmunoassay), immunoprecipitation, FIA (fluorescence immunoassay), FPIA (fluorescence polorization immunoassay), CIA (chemiluminescent immunoassay), electochemical detection.

37. The method of claim 34, wherein the subject is a mammal.

38. The method of claim 37, wherein the subject is a human.

39. The method of claim 34 wherein the biological sample is selected from the group consisting of: blood, serum, plasma, urine, semen, seminal fluid, seminal plasma, prostatic fluid, pre-ejaculatory fluid (Cowper's fluid), excreta, tears, saliva, sweat, biopsy, ascites, cerebrospinal fluid, lymph, and tissue extract sample.

40. The method of claim 39 wherein the biological sample is a plasma or urine sample.

41. A method for the in vitro differential diagnosis of healthy, colorectal cancer, benign disease of the large intestines, precancerous lesion of the large intestine, localized cancer of the large intestine, metastasised colorectal cancer, and acute or chronic inflammation of the large intestines in a subject, comprising detection of one or more differentially expressed biomarkers in a biological sample by:
(a) obtaining the biological sample from the subject;
(b) contacting said sample with one or more binding molecules specific for SEQ ID NO:1; and
(c) detecting the quantity, presence or absence of said one or more biomarker;
wherein the presence or absence of said biomarker(s) allows for the differential diagnosis of the subject as healthy, having benign disease of the large intestines, precancerous lesion of the large intestines, localized cancer of the large intestine, metastasised colorectal cancer, and/or having acute or chronic inflammation of the large intestine, or as being healthy; and
initiating a treatment for colorectal cancer if the subject is classified as having colorectal cancer.

42. The method of claim 41 wherein the detecting is done by an immunosorbent assay.

43. A kit for the diagnosis of colorectal cancer within a subject comprising a solution, one or more binding molecule(s), a detection substrate, and instructions, wherein the instructions outline in detail the method of claim 41.

44. A polypeptide consisting of the amino acids EIPPPPPED FPLPPPPLAG DGDDAEGALG GAFPPPPPPI EESFPPAPLE (SEQ ID NO:1).

45. A composition for treating a disease of the large intestine comprising a molecular entity which modulates the polypeptide of claim 44 and a pharmaceutically acceptable carrier.

46. A composition as claimed in claim 45, wherein said disease of the large intestine is selected from the group consisting of colorectal cancer and non-malignant disease of the large intestines.

47. A composition as claimed in claim 46, wherein said disease of the large intestine is selected from the group consisting of benign disease of the large intestines, precancerous lesion of the large intestine, localized cancer of the large intestine, metastasised colorectal cancer, and acute or chronic inflammation of the large intestines.

48. A composition as claimed in claim 47, wherein said molecular entity is selected from the group consisting of nucleotides, oligonucleotides, polynucleotides, amino acids, peptides, polypeptides, proteins, antibodies, immunoglobulins, small organic molecules, pharmaceutical agents, agonists, antagonists, derivatives or combinations thereof.

49. The use of a composition as claimed in claim 45 for treating a disease of the large intestine.

50. The use of claim 49, wherein said disease of the large intestine is selected from the group consisting of colorectal cancer and non-malignant disease of the large intestines.

51. A pharmaceutical composition comprising the polypeptide of claim 44 in a pharmaceutically acceptable carrier.

52. A method of treating colorectal cancer comprising administration of a therapeutically acceptable amount of the pharmaceutical composition of claim 51.

53. An antibody having specificity for the polypeptide of claim 44.
